(19) United States
(12) Patent Application Publication
(10) Pub. No.: US 2009/0232868 A1
(43) Pub. Date: Sep. 17, 2009

(54) Nitric Oxide Releasing Polymer Composition

(75) Inventors: Mingfei Chen, Santa Rosa, CA (US); Peiwen Cheng, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/049,648

(22) Filed: Mar. 17, 2008

(51) Int. Cl. A61K 9/00; C08F 118/02; A61K 39/395; A61K 31/7052; A61K 31/663; A61K 31/4353; A61F 2/82

(52) U.S. Cl. 424/423; 526/319; 424/130.1; 514/108; 514/291; 623/1.42; 623/1.13; 623/1.26; 514/44 R; 514/44 A

(57) ABSTRACT

Disclosed herein are biocompatible carbon-based nitric oxide (NO) donating polymers suitable for forming and coating medical devices. These polymers have acrylate backbones and are comprised of substantially hydrophobic monomers. The NO donating polymers are carbon based wherein the diazeniumdiolate group is attached to the acetate group on an acetate based monomer. Incorporating a vinyl acetate monomer into an acrylate based polymer allows diazeniumdiolation of a polymer that would otherwise not accommodate the diazeniumdiolate group.

### FIELD OF THE INVENTION

The present invention relates to nitric oxide donating polymers and copolymers suitable for the coating and fabricating of implantable medical devices.

### BACKGROUND OF THE INVENTION

Nitric oxide (NO) is a simple diatomic molecule that plays a diverse and complex role in cellular physiology. Nitric oxide is typically known as a component of automobile exhaust that is a precursor in the formation of photochemical smog. Therefore, NO is commonly associated with the brownish air that accumulates over metropolitan areas all over the world. However, NO is not always associated with adverse environmental processes. In fact, as a result of the pioneering work of Ferid Murad et al. it is now known that NO is a powerful signaling compound and cytotoxic/cytostatic agent found in nearly every tissue including endothelial cells, neural cells and macrophages. Mammalian cells synthesize NO using a two step enzymatic process that oxidizes L-arginine to N-ω-hydroxy-L-arginine, which is then converted into L-citrulline and an uncharged NO free radical. Three different nitric oxide synthase enzymes regulate NO production. Neuronal nitric oxide synthase (NOSI, or nNOS) is formed within neuronal tissue and plays an essential role in neurotransmission; endothelial nitric oxide synthase (NOS3 or eNOS), is secreted by endothelial cells and induces vasodilatation; inducible nitric oxide synthase (NOS2 or iNOS) is principally found in macrophages, hepatocytes and chondrocytes and is associated with immune cytotoxicity.

Neuronal NOS and eNOS are constitutive enzymes that regulate the rapid, short-term release of small amounts of NO. In these minute amounts, NO activates guanylate cyclase which elevates cyclic guanosine monophosphate (cGMP) concentrations which in turn increase intracellular Ca²⁺ levels. Increased intracellular Ca²⁺ concentrations result in smooth muscle relaxation which accounts for NO's vasodilating effects. Inducible NOS is responsible for the sustained release of larger amounts of NO and is activated by extracellular factors including endotoxins and cytokines. These higher NO levels play a key role in cellular immunity.

Medical research is rapidly discovering therapeutic applications for NO including the fields of vascular surgery and interventional cardiology. Procedures used to clear blocked arteries such as percutaneous transluminal coronary angioplasty (PTCA) (also known as balloon angioplasty) and atherectomy and/or stent placement can result in vessel wall injury at the site of balloon expansion or stent deployment. In response to this injury a complex multi-factorial process known as restenosis can occur whereby the previously opened vessel lumen narrows and becomes re-occluded. Restenosis is initiated when thrombocytes (platelets) migrating to the injury site release mitogens into the injured endothelium. Thrombocytes begin to aggregate and adhere to the injury site initiating thrombogenesis, or clot formation. As a result, the previously opened lumen begins to narrow as thrombocytes and fibrin collect on the vessel wall. In a more frequently encountered mechanism of restenosis, the mitogens secreted by activated thrombocytes adhering to the vessel wall stimulate overproliferation of vascular smooth muscle cells during the healing process, restricting or occluding the injured vessel lumen. The resulting neointimal hyperplasia is the major cause of a stent restenosis.

Recently, NO has been shown to significantly reduce thrombocyte aggregation and adhesion; this combined with NO's directly cytotoxic/cytostatic properties may significantly reduce vascular smooth muscle cell proliferation and help prevent restenosis. Thrombocyte aggregation occurs within minutes following the initial vascular insult and once the cascade of events leading to restenosis is initiated, irreparable damage can result. Moreover, the risk of thrombogenesis and restenosis persists until the endothelium lining the vessel lumen has been repaired. Therefore, it is essential that NO, or any anti-restenotic agent, reach the injury site immediately.

One approach for providing a therapeutic level of NO at an injury site is to increase systemic NO levels prophylactically. This can be accomplished by stimulating endogenous NO production or using exogenous NO sources. Methods to regulate endogenous NO release have primarily focused on activation of synthetic pathways using excess amounts of NO precursors like L-arginine, or increasing expression of nitric oxide synthase (NOS) using gene therapy. U.S. Pat. Nos. 5,945,452, 5,891,459 and 5,428,070 describe sustained NO elevation using orally administrated L-arginine and/or L-lysine. However, these methods have not been proven effective in preventing restenosis. Regulating endogenously expressed NO using gene therapy techniques remains highly experimental and has not yet proven safe and effective. U.S. Pat. Nos. 5,268,465, 5,468,630 and 5,658,565, describe various gene therapy approaches.

Exogenous NO sources such as pure NO gas are highly toxic, short-lived and relatively insoluble in physiological fluids. Consequently, systemic exogenous NO delivery is generally accomplished using organic nitrate prodrugs such as nitroglycerin tablets, intravenous suspensions, sprays and transdermal patches. The human body rapidly converts nitroglycerin into NO; however, enzyme levels and co-factors required to activate the prodrug are rapidly depleted, resulting in drug tolerance. Moreover, systemic NO administration can have devastating side effects including hypotension and free radical cell damage. Therefore, using organic nitrate prodrugs to maintain systemic anti-restenotic therapeutic blood levels is not currently possible.

Therefore, considerable attention has been focused on localized, or site specific, NO delivery to ameliorate the disadvantages associated with systemic prophylaxis. Implantable medical devices and/or local gene therapy techniques including medical devices coated with NO-releasing compounds, or vectors that deliver NOS genes to target cells, have been evaluated. Like their systemic counterparts, gene therapy techniques for the localized NO delivery have not been proven safe and effective. There are still significant technical hurdles and safety concerns that must be overcome before site-specific NOS gene delivery will become a reality.

However, significant progress has been made in the field of localized exogenous NO application. To be effective at preventing restenosis an inhibitory therapeutic such as NO must be administered for a sustained period at therapeutic levels. Consequently, any NO releasing medical device used to treat restenosis must be suitable for implantation. An ideal candidate device is the vascular stent. Therefore, a stent that safely provides therapeutically effective amounts of NO to a precise location would represent a significant advance in restenosis treatment and prevention.

Nitric oxide-releasing compounds suitable for in vivo applications have been developed by a number of investigators. As early as 1960 it was demonstrated that nitric oxide gas could be reacted with amines, for example, diethylamine, to form NO-releasing anions having the following general formula R—R′N—N(O)NO. Salts of these compounds could spontaneously decompose and release NO in solution.

Nitric oxide-releasing compounds with sufficient stability at body temperatures to be useful as therapeutics were ultimately developed by Keefer et al. as described in U.S. Pat. Nos. 4,954,526, 5,039,705, 5,155,137, 5,212,204, 5,250,550, 5,366,997, 5,405,919, 5,525,357 and 5,650,447, all of which are herein incorporated by reference.

The in vivo half-life of NO, however, is limited, causing difficulties in delivering NO to the intended area. Therefore NO-releasing compounds which can produce extended release of NO are needed. Several exemplary NO-releasing compounds have been developed for this purpose, including for example a NO donating aspirin derivative, amyl nitrite and isosorbide dinitrate. Additionally, biocompatible polymers having NO adducts (see, for example, U.S. Patent Publications 2006/0008529 and 2004/0037836) that release NO in a controlled manner have been reported.

Secondary amines have the ability to bind two NO molecules and release them in an aqueous environment. Exposing secondary amines to basic conditions while incorporating NO gas under high pressure leads to the formation of nitrogen-based diazeniumdiolates.

However, nitrogen-based diazeniumdiolate-containing polymers cannot be formulated as bioabsorbable polymers due to the breakdown of the nitrogen-based diazeniumdiolate moiety into nitrosamines which are carcinogens and irritants. Therefore bioabsorbable NO donating polymers that do not incorporate nitrogen-based diazeniumdiolates are needed. The present invention provides carbon-based NO donating polymers.

### SUMMARY OF THE INVENTION

Disclosed herein are biocompatible carbon-based diazeniumdiolate nitric oxide (NO) donating polymers suitable for forming and coating medical devices. In one embodiment, a nitric oxide donating polymer is described comprising at least one polymerizable monomer selected from the group comprising n-butyl methacrylate, n-hexyl methacrylate, cyclohexyl methacrylate, 2-ethylhexyl methacrylate, and; at least one vinyl monomer comprising at least one acetate group; and wherein said acetate group binds at least one diazeniumdiolate group. On one embodiment, the polymer comprises Formula 1:

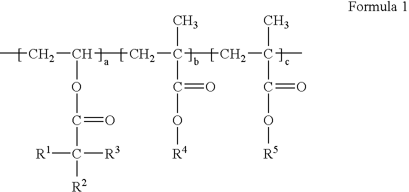

wherein R⁴ and R⁵ are independently selected from the group comprising C₁ to C₂₀ straight chain alkyls, C₃ to C₈ cycloalkyls, C₂ to C₂₀ alkenyls, C₂ to C₂₀ alkynyls, C₂ to C₁₄ heteroatom substituted alkyls, C₂ to C₁₄ heteroatom substituted cycloalkyls, C₁ to C₁₀ multiple amine containing-hydrocarbons, C₄ to C₁₀ substituted aryls and C₄ to C₁₀ substituted heteroatom substituted heteroaryls; R¹, R² and R³ are independently a hydrogen or said diazeniumdiolate group; a, b, and c are respectively 1-2000, 1-2000, and 1-2000.

In one embodiment, the polymer comprises Formula 3:

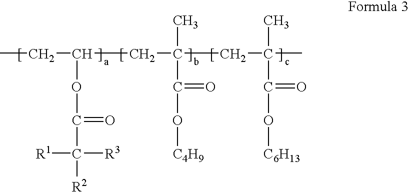

wherein R¹, R² and R³ are independently a hydrogen or said diazeniumdiolate group; a, b, and c are respectively 1-2000, 1-2000, and 1-2000.

In one embodiment, the polymer has the general structure of Formula 5:

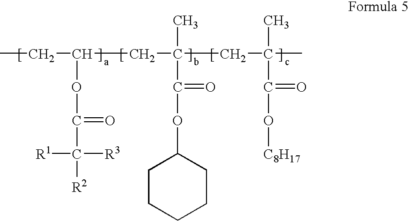

wherein R¹, R² and R³ are independently a hydrogen or said diazeniumdiolate group; a, b, and c are respectively 1-2000, 1-2000, and 1-2000.

In one embodiment, the vinyl monomer is vinyl acetate. In another embodiment, the polydispersity index is between 1.1 and 5.0. In one embodiment, the glass transition temperature is between −30 and 150 C.

In one embodiment, a medical device is described having a coating comprised of said nitric oxide donating polymer as described above. In one embodiment, the implantable medical device is selected from the group consisting of vascular stents, shunts, vascular grafts, stent grafts, heart valves, catheters, pacemakers, pacemaker leads, bile duct stents and defibrillators. In one embodiment, the polymer can release at least one drug in addition to nitric oxide. In another embodiment, the at least one drug is selected from the group consisting of anti-proliferatives, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids. In another embodiment, the drug comprises at least one compound selected from the group consisting of sirolimus (rapamycin), tacrolimus (FK506), everolimus (certican), temsirolimus (CCI-779) and zotarolimus (ABT-578).

In one embodiment, a medical device is described having a structure wherein said structure comprising said nitric oxide donating polymer described above. In another embodiment, the implantable medical device is selected from the group consisting of vascular stents, shunts, vascular grafts, stent grafts, heart valves, catheters, pacemakers, pacemaker leads, bile duct stents and defibrillators. In another embodiment, the polymer can release at least one drug in addition to nitric oxide.

In one embodiment, the at least one drug is selected from the group consisting of anti-proliferatives, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids. In another embodiment, the drug comprises at least one compound selected from the group consisting of sirolimus (rapamycin), tacrolimus (FK506), everolimus (certican), temsirolimus (CCI-779) and zotarolimus (ABT-578).

### Definition of Terms

Backbone: As used herein “backbone” refers to the main chain of a polymer or copolymer of the present invention.

Biocompatible: As used herein “biocompatible” shall mean any material that does not cause injury or death to the animal or induce an adverse reaction in an animal when placed in intimate contact with the animal's tissues. Adverse reactions include inflammation, infection, fibrotic tissue formation, cell death, or thrombosis.

Biodegradable: As used herein “biodegradable” refers to the polymer or copolymer of the present invention being biocompatible and subject to in vivo breakdown through the action of normal biochemical pathways. From time-to-time bioresorbable and biodegradable may be used interchangeably, however they are not coextensive. Biodegradable polymers may or may not be reabsorbed into surrounding tissues, however all bioresorbable polymers are considered biodegradable. The biodegradable polymers of the present invention are capable of being cleaved into biocompatible byproducts through chemical- or enzyme-catalyzed hydrolysis.

Copolymer: As used herein “copolymer” refers to a macromolecule produced by the simultaneous or stepwise polymerization of two or more dissimilar monomeric units. Copolymer shall include, but not be limited to, bipolymers (two dissimilar units), terpolymer (three dissimilar units), etc.

Diazeniumdiolate: As used herein in relation to the present invention, unless specifically stated otherwise, “diazeniumdiolate” refers to carbon based diazeniumdiolate groups as opposed to nitrogen based diazeniumdiolate groups commonly presented in the art. Diazeniumdiolate groups as used herein shall have the common structure seen below.

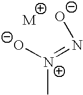

The bond from the positively charged quaternary amine is the bonding point between the diazeniumdiolate and the substrate of interest. M is an appropriate counter ion selected from the group comprising Na⁺, K⁺, Li⁺, Ca²⁺, Zn²⁺, Fe²⁺ and Fe³⁺.

Drug: As used herein, “bioactive agent” shall include any compound or drug having a therapeutic effect in an animal. Exemplary, non limiting examples include anti-proliferatives including, but not limited to, macrolide antibiotics including FKBP-12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids. Drugs can also refer to bioactive agents including anti-proliferative compounds, cytostatic compounds, toxic compounds, anti-inflammatory compounds, chemotherapeutic agents, analgesics, antibiotics, protease inhibitors, statins, nucleic acids, polypeptides, growth factors and delivery vectors including recombinant micro-organisms, liposomes, and the like.

Exemplary FKBP-12 binding agents include sirolimus (rapamycin), tacrolimus (FK506), everolimus (certican or RAD-001), temsirolimus (CCI-779 or amorphous rapamycin 42-ester with 3-hydroxy-2-(hydroxymethyl)-2-methylpropionic acid as disclosed in U.S. patent application Ser. No. 10/930,487) and zotarolimus (ABT-578; see U.S. Pat. Nos. 6,015,815 and 6,329,386). Additionally, other rapamycin hydroxyesters as disclosed in U.S. Pat. No. 5,362,718 may be used in combination with the polymers of the present invention.

Ductility: As used herein “ductility, or ductile” is a polymer attribute characterized by the polymer's resistance to fracture or cracking when folded, stressed or strained at operating temperatures. When used in reference to the polymer coating compositions of the present invention the normal operating temperature for the coating will be between room temperature and body temperature or approximately between 15° C. and 40° C. Polymer durability in a defined environment is often a function of its elasticity/ductility.

Functional Side Chain: As used herein “functional side chain” encompasses a first chemical constituent(s) typically capable of binding to a second chemical constituent(s), wherein the first chemical constituent modifies a chemical or physical characteristic of the second chemical constituent. Functional groups associated with the functional side chains include acetyl groups, vinyl groups, hydroxyl groups, oxo groups, carboxyl groups, thiol groups, amino groups, phosphor groups and others known to those skilled in the art and as depicted in the present specification and claims.

Glass Transition Temperature: As used herein “glass transition temperature,” abbreviated (T_{g}) herein, refers to a temperature wherein a polymer structurally transitions from a elastic pliable state to a rigid and brittle state.

Hydrophilic: As used herein “hydrophilic” refers to a substance that has solubility in water of more than 200 micrograms per milliliter.

Hydrophobic: As used herein “hydrophobic” refers to a substance that has solubility in water of less than 200 micrograms per milliliter.

Kinetics: The drug release “kinetics” of the present invention should be either zero-order or a combination of first and zero order.

Mₙ: As used herein Mₙ refers to number-average molecular weight. Mathematically it is represented by the following formula:

$${M}_{n} = {\sum }_{i} {N}_{i} {M}_{i} / {\sum }_{i} {N}_{i} ,$$

wherein the Nᵢ is the number of moles whose weight is Mᵢ.

M_{w}: As used herein M_{w} refers to weight average molecular weight that is the average weight that a given polymer may have. Mathematically it is represented by the following formula:

$${M}_{w} = {\sum }_{i} {N}_{i} {M}_{i}^{2} / {\sum }_{i} {N}_{i} {M}_{i} ,$$

wherein Nᵢ the number of molecules whose weight is Mᵢ.

Polydispersity Index: As used herein “polydispersity index,” abbreviated PDI herein, refers to the weight distribution of polymers in a sample. The polydispersity index is the fraction of the weight average molecular weight to the number-average molecular weight. Mathematically, it is represented by the following formula: PDI=M_{w}/Mₙ.

### BRIEF DESCRIPTION OF THE DRAWINGS

FIG. 1 depicts nitric oxide release from the diazeniumdiolated C153-1688-95-1 polymer.

### DETAILED DESCRIPTION OF THE INVENTION

Disclosed herein are biocompatible carbon-based diazeniumdiolate nitric oxide (NO) donating polymers suitable for forming and coating medical devices. The polymers have acrylate backbones and are comprised of substantially hydrophobic monomers. The polymers have the general structure of Formula 1.

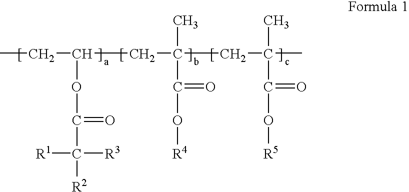

In one embodiment, the polymer backbone is substantially acrylate based and wherein at least one of R¹, R², or R³ is a diazeniumdiolate group. The groups R⁴ and R⁵ are independently selected from the group comprising C₁ to C₂₀ straight chain alkyls, C₃ to C₈ cycloalkyls, C₂ to C₂₀ alkenyls, C₂ to C₂₀ alkynyls, C₂ to C₁₄ heteroatom substituted alkyls, C₂ to C₁₄ heteroatom substituted cycloalkyls, C₄ to C₁₀ substituted aryls and C₄ to C₁₀ substituted heteroatom substituted heteroaryls. The acetate group's alpha carbon can be diazeniumdiolated on any three of its hydrogen, therefore, R¹, R², and R³ can independently be a diazeniumdiolate group or hydrogen.

In one embodiment, a, b and c of Formula 2 are individually integers ranging from 1 to 20,000.

The NO donating polymers are carbon based wherein the diazeniumdiolate group is attached to the acetate group on an acetate based monomer. Incorporating a vinyl acetate monomer into an acrylate based polymer allows diazeniumdiolation of a polymer that would otherwise not accommodate the diazeniumdiolate group.

The polymer backbone comprises monomers including, but not limited to, vinyl acetate, n-butyl methacrylate, and n-hexyl methacrylate, cyclohexyl methacrylate, 2-ethylhexyl methacrylate, methyl methacrylate, ethyl methacrylate, propyl methacrylate, pentyl methacrylate, octyl methacrylate, lauryl methacrylate and 2-ethoxyethyl methacrylate, 2-hydroxyethyl methacrylate, hydroxypropyl methacrylate, methyl acrylate, ethyl acrylate, propyl acrylate, butyl acrylate, pentyl acrylate, hexyl acrylate and 2-ethylhexyl acrylate, octyl acrylate, lauryl acrylate, 2-hydroxyethyl acrylate and hydroxypropyl acrylate.and combinations thereof.

The monomers described herein are either commercially available or synthesized with well known synthetic transformations.

In one embodiment, the polymerization of vinyl acetate, n-butyl methacrylate and n-hexyl methacrylate forms the terpolymer of Formula 2. These monomers are polymerized, in a non-limiting example, in the presence of an initiator such as, but not limited to azobisisobutyronitrile (AIBN). The polymer represented by Formula 2 can be diazeniumdiolated as described herein.

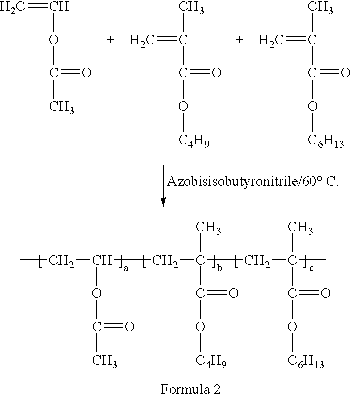

In one embodiment, a, b and c of Formula 2 are individually integers ranging from 1 to 20,000.

In one embodiment, the polymer of Formula 2 is diazeniumdiolated to form the polymer of Formula 3 wherein R¹, R² and R³ are individually hydrogen or a diazeniumdiolate group.

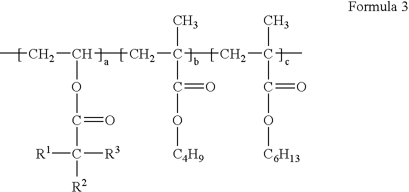

In another embodiment, the polymerization of vinyl acetate, cyclohexyl methacrylate and 2-ethylhexyl methacrylate forms the terpolymer of Formula 4. These monomers are polymerized, in a non-limiting example, in the presence of an initiator such as, but not limited to AIBN. The polymer represented by Formula 4 can be diazeniumdiolated as described herein.

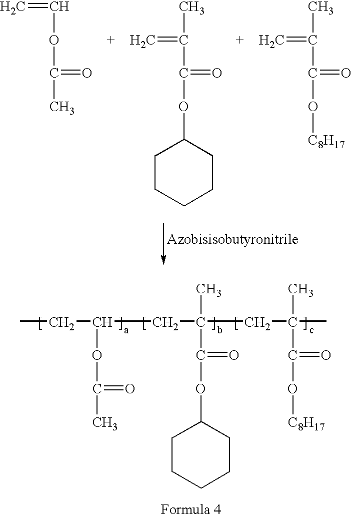

In one embodiment, a, b and c of Formula 4 are individually integers ranging from 1 to 20,000.

In another embodiment, the polymer of Formula 4 is diazeniumdiolated to form the polymer of Formula 5 wherein R¹, R² and R³ are individually hydrogen or a diazeniumdiolate group.

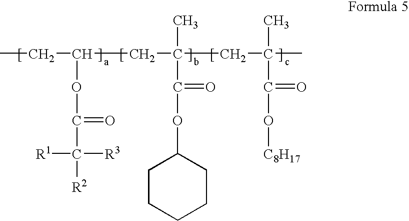

Medical devices, including implantable medical devices, are fabricated and/or coated with the polymers, therefore, the physical properties of the polymers are considered in light of the specific application at hand. The physical properties of the polymers can be fine tuned so that the polymers can optimally perform for their intended use. Properties that can be fine tuned, without limitation, include T_{g}, molecular weight (both Mₙ and M_{w}), polydispersity index (PDI, the quotient of M_{w}/Mₙ), degree of elasticity and degree of amphiphilicity. In one embodiment, the T_{g} of the polymers range from about −30° C. to about 150° C. In still another embodiment, the PDI of the polymers range from about 1.1 to about 5.0. In another embodiment, the T_{g} of the polymers ranges form about 5° C. to about 50° C. In still another embodiment, the PDI of the polymers range from about 1.5 to about 3.0.

Also taken into account is fine tuning, or modifying, the glass transition temperature (T_{g}) of the biostable NO donating polymers. Drug elution from polymers depends on many factors including density, the drug to be eluted, molecular composition of the polymer and T_{g}. Higher T_{g}s, for example temperatures above 40° C., result in more brittle polymers while lower T_{g}s, e.g lower than 40° C., result in more pliable and elastic polymers at higher temperatures. Drug elution is slow from polymers that have high T_{g}s while faster rates of drug elution are observed with polymers possessing low T_{g}s. In one embodiment, the T_{g} of the polymer is selected to be lower than 37° C.

In one embodiment, the polymers can be used to form and coat medical devices. Coating polymers having relatively high T_{g}s can result in medical devices with unsuitable drug eluting properties as well as unwanted brittleness. In the cases of polymer-coated vascular stents, a relatively low T_{g} in the coating polymer effects the deployment of the vascular stent. For example, polymer coatings with low T_{g}s are “sticky” and adhere to the balloon used to expand the vascular stent during deployment, causing problems with the deployment of the stent. Low T_{g} polymers, however, have beneficial features in that polymers having low T_{g}s are more elastic at a given temperature than polymers having higher T_{g}s. Expanding and contracting a polymer-coated vascular stent mechanically stresses the coating. If the coating is too brittle, i.e. has a relatively high T_{g}, then fractures may result in the coating possibly rendering the coating inoperable. If the coating is elastic, i.e has a relatively low T_{g}, then the stresses experienced by the coating are less likely to mechanically alter the structural integrity of the coating. Therefore, the T_{g}s of the polymers can be fine tuned for appropriate coating applications by a combination of monomer composition and synthesis conditions. The polymers are engineered to have adjustable physical properties enabling the practitioner to choose the appropriate polymer for the function desired.

In order to tune, or modify, the polymers, a variety of properties are considered including, but not limited to, T_{g}, connectivity, molecular weight and thermal properties.

The NO donating polymers donate NO once exposed to a physiological environment. The rates of NO release from the polymers can be fine tuned by selecting the appropriate monomer ratios and diazoniumdiolate stabilizing counterion selection.

Medical devices, including implantable medical devices, are fabricated and/or coated with the polymers, therefore, the physical properties of the polymers are considered in light of the specific application at hand. The physical properties of the polymers can be fine tuned so that the polymers can optimally perform for their intended use. Properties that can be fine tuned, without limitation, include T_{g}, molecular weight (both Mₙ and M_{w}), polydispersity index (PDI, the quotient of M_{w}/Mₙ), degree of elasticity and degree of amphiphlicity. In one embodiment, the T_{g} of the polymers range from about −30° C. to about 150° C. In still another embodiment, the PDI of the polymers range from about 1.1 to about 5.0. In another embodiment, the T_{g} of the polymers ranges form about 5° C. to about 50° C. In still another embodiment, the PDI of the polymers range from about 1.5 to about 3.0.

Implantable medical devices suitable for coating with the NO donating polymers include, but are not limited to, vascular stents, stent grafts, urethral stents, bile duct stents, catheters, guide wires, pacemaker leads, bone screws, sutures and prosthetic heart valves. The polymers are suitable for fabricating implantable medical devices. Medical devices which can be manufactured from the NO donating polymers include, but are not limited to, vascular stents, stent grafts, urethral stents, bile duct stents, catheters, guide wires, pacemaker leads, bone screws, sutures and prosthetic heart valves.

The polymers are intended for medical devices deployed in a hemodynamic environment and possess excellent adhesive properties. That is, the coating must be biocompatible and stably linked to the medical device surface. Many different materials can be used to fabricate the implantable medical devices including, but not limited to, stainless steel, nitinol, aluminum, chromium, titanium, gold, cobalt, ceramics, and a wide range of synthetic polymeric and natural materials including, but not limited to, collagen, fibrin and plant fibers. All of these materials, and others, may be used with the polymers made in accordance with the teachings described herein. Furthermore, the polymers can be used to fabricate an entire medical device. The medical device or polymer coating may or may not be bioerodable.

There are many theories that attempt to explain, or contribute to our understanding of how polymers adhere to surfaces. The most important forces include electrostatic and hydrogen bonding. However, other factors including wettability, absorption and resiliency also determine how well a polymer will adhere to different surfaces. Therefore, polymer base coats, or primers are often used in order to create a more uniform coating surface.

The NO donating polymers can be applied to medical device surfaces, either primed or bare, in any manner known to those skilled in the art. Compatible application methods include, but are not limited to, spraying, dipping, brushing, vacuum-deposition, electrostatic spray coating, plasma coating, spin coating electrochemical coating, and others.

Moreover, the NO donating polymers may be used with a cap coat. A cap coat as used herein refers to the outermost coating layer applied over another coating. The NO donating polymer coating is applied over the primer coat. Then, a polymer cap coat is applied over the NO donating polymeric coating. The cap coat may optionally serve as a diffusion barrier to control NO release. The cap coat may be merely a biocompatible polymer applied to the surface of the sent to protect the stent and have no effect on NO release rates. In one embodiment, a hydrophilic cap coat may be applied to enhance biocompatibility of the otherwise hydrophobic acrylate polymer.

The NO donating polymers are also useful for the delivery and controlled release of drugs. Drugs that are suitable for release from the polymers include, but are not limited to, anti-proliferative compounds, cytostatic compounds, toxic compounds, anti-inflammatory compounds, chemotherapeutic agents, analgesics, antibiotics, protease inhibitors, statins, nucleic acids, polypeptides, growth factors and delivery vectors including recombinant micro-organisms, liposomes, and the like.

In one embodiment the drugs controllably released include, but are not limited to, macrolide antibiotics including FKBP-12 binding agents. Exemplary drugs of this class include sirolimus (rapamycin), tacrolimus (FK506), everolimus (certican or RAD-001), temsirolimus (CCI-779 or amorphous rapamycin 42-ester with 3-hydroxy-2-(hydroxymethyl)-2-methylpropionic acid as disclosed in U.S. patent application Ser. No. 10/930,487) and zotarolimus (ABT-578; see U.S. Pat. Nos. 6,015,815 and 6,329,386). Additionally, other rapamycin hydroxyesters as disclosed in U.S. Pat. No. 5,362,718 may be used in combination with the polymers. The entire contents of all of preceding patents and patent applications are herein incorporated by reference for all they teach related to FKBP-12 binding compounds and the derivatives.

### EXAMPLES

The following non-limiting examples provide methods for the synthesis of exemplary polymers.

### Example 1

### Synthesis of Copolymers of vinyl acetate, n-butyl methacrylate, and n-hexyl methacrylate

All chemicals were charged to a 120 mL glass bottle according to Table 1. The bottles were sealed with a silicone septum and purged with nitrogen for 20 minutes. The bottles were heated in an oil bath to 60° C. At the end of the reaction, the polymers were purified by precipitation and reprecipitation (from dichloromethane solution) into methanol. The polymers were characterized by ¹H nuclear magnetic resonance spectroscopy (NMR), gel permeation chromatography (GPC) and differential scanning calorimetry (DSC). The properties of the polymers are listed in Table 2.

TABLE 1
Polymerization Formulation

| Entry | BMA (g) | HMA (g) | VA (g) | AIBN (g) | Reaction Time (h) |
|---|---|---|---|---|---|
| 1 | 7.50 | 4.50 | 17.98 | 0.1131 | 5.5 |
| 2 | 5.63 | 3.38 | 21.01 | 0.1132 | 5.5 |
| 3 | 3.75 | 2.28 | 24.01 | 0.1130 | 5.5 |
| 4 | 1.88 | 1.18 | 27.03 | 0.1131 | 4.0 |

VA = vinyl acetate;
BMA = n-butyl methacrylate;
HMA = n-hexyl methacrylate;
AIBN = 2,2′-azobisisobutyronitrile

TABLE 2
Properties of Copolymers of VA/BMA/HMA

| | | Polymer | | | |
|---|---|---|---|---|---|
| | | Composition | | | |
| | | VA/BMA/HMA | | | |
| Entry | Polymer Code | (mol %) | Mₙ | PDI | T_{g} (° C.) |
| 1 | C146-1688-083-1 | 13/58/29 | 89493.0 | 1.95 | 6.8 |
| 2 | C147-1688-083-2 | 17/55/28 | 78173.5 | 1.84 | 11.7 |
| 3 | C148-1688-083-3 | 27/49/24 | 71172.5 | 1.68 | 15.8 |
| 4 | C149-1688-083-4 | 41/39/20 | 78173.5 | 1.84 | 17.6 |

1 - estimated

### Example 2

### Synthesis of Copolymers of vinyl acetate, cyclohexyl methacrylate and 2-ethylhexyl methacrylate

All chemical reagents were charged to a 120 mL glass bottle according to Table 3. The bottles were sealed with a silicone septum and purged with nitrogen for 20 minutes. The bottles were heated to 60° C. in an oil bath. At the end of the reaction, the copolymers were purified by precipitation and reprecipitation (from dichloromethane solution) into methanol. The polymers were characterized by ¹H NMR, GPC and DSC. The properties of the polymers are listed in Table 4.

TABLE 3
Polymerization Formulation

| Entry | CMA (g) | OMA (g) | VA (g) | AIBN (g) | Reaction Time (h) |
|---|---|---|---|---|---|
| 1 | 3.61 | 8.40 | 18.01 | 0.1131 | 5.0 |
| 2 | 2.70 | 6.27 | 21.00 | 0.1131 | 5.0 |
| 3 | 1.79 | 4.19 | 24.10 | 0.1131 | 5.0 |
| 4 | 0.90 | 2.11 | 26.98 | 0.1131 | 5.0 |

CMA = cyclohexyl methacrylate;
OMA = 2-ethylhexyl methacrylate;
VA = vinyl acetate;
AIBN = 2,2′-azobisisobutyronitrile

TABLE 4
Properties of Copolymers of VA/OMA/CMA

| | | Polymer | | | |
|---|---|---|---|---|---|
| | | Composition | | | |
| | | VA/OMA/CMA | | | |
| Entry | Polymer Code | (mol %) | Mn | PDI | Tg (° C.) |
| 1 | C153-1688-95-1 | 29/56/15 | 225962 | 1.94 | 14.9 |
| 2 | C154-1688-95-2 | 28/52/20 | 187580 | 2.03 | 17.8 |
| 3 | C155-1688-95-3 | 25/47/28 | 160937 | 1.89 | 21.3 |
| 4 | C156-1688-95-4 | 22/36/42 | 147514 | 1.78 | 22.5 |

### Example 3

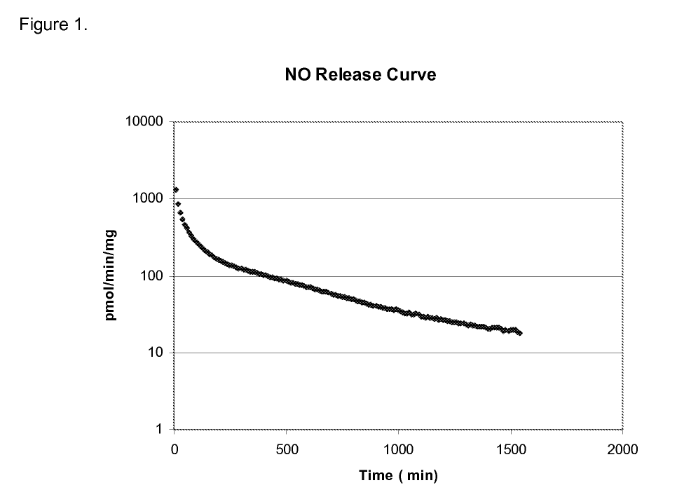

5.0 g of polymer C153-1688-95-1 disclosed in Example 2 was dissolved in 100 mL anhydrous THF and 50 mL 1M sodium trimethylsiloanate in THF was added. The reactor was purged with argon to remove oxygen. The reactor was then pressurized with 80 psi nitric oxide gas overnight. The diazeniumdiolated polymer was purified by precipitation in ethanol and washed with THF/ethanol (v/v 1:1). The polymer was dried in vacuum at room temperature. The dry polymer was dissolved in THF/methanol (v/v 1:1) to make 1% solution. Stainless coupon was dip coated with the NO-releasing polymer and analyzed for nitric oxide release in phosphate buffer at pH=7.4 with a chemiluminescence NO analyzer. The NO release is illustrated in FIG. 1.

### Example 4

The diazeniumdioated polymer from example 3 was re-dissolved in methanol/THF (v/v 1:1) and sprayed onto 3.0×18 mm Medtronic Driver® stents. The stents were further cap coated with un-diazeniumdiolated C153-1688-95-1 polymer.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

I claim:

1. A nitric oxide donating polymer comprising:
at least one polymerizable monomer selected from the group comprising n-butyl methacrylate, n-hexyl methacrylate, cyclohexyl methacrylate, 2-ethylhexyl methacrylate, and; at least one vinyl monomer comprising at least one acetate group; and wherein said acetate group binds at least one diazeniumdiolate group.

2. The nitric oxide donating polymer of claim 1 wherein said polymer comprises Formula 1: 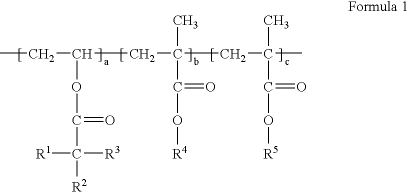
wherein R⁴ and R⁵ are independently selected from the group comprising C₁ to C₂₀ straight chain alkyls, C₃ to C₈ cycloalkyls, C₂ to C₂₀ alkenyls, C₂ to C₂₀ alkynyls, C₂ to C₁₄ heteroatom substituted alkyls, C₂ to C₁₄ heteroatom substituted cycloalkyls, C₁ to C₁₀ multiple amine containing-hydrocarbons, C₄ to C₁₀ substituted aryls and C₄ to C₁₀ substituted heteroatom substituted heteroaryls; R¹, R² and R³ are independently a hydrogen or said diazeniumdiolate group; a, b, and c are respectively 1-2000, 1-2000, and 1-2000.

3. The nitric oxide donating polymer of claim 1 wherein said polymer comprises Formula 3: 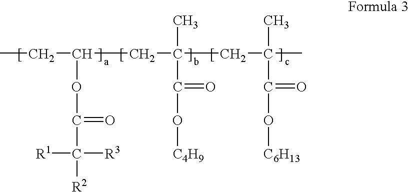
wherein R¹, R² and R³ are independently a hydrogen or said diazeniumdiolate group; a, b, and c are respectively 1-2000, 1-2000, and 1-2000.

4. The nitric oxide donating polymer of claim 1 wherein said polymer has the general structure of Formula 5: 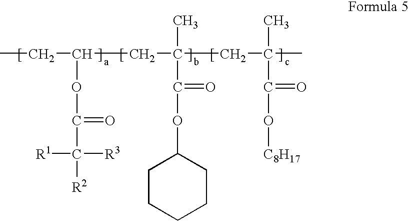
wherein R¹, R² and R³ are independently a hydrogen or said diazeniumdiolate group; a, b, and c are respectively 1-2000, 1-2000, and 1-2000.

5. The nitric oxide donating polymer of claim 1 wherein said vinyl monomer is vinyl acetate.

6. The nitric oxide donating polymer of claim 1 wherein the polydispersity index is between 1.1 and 5.0.

7. The nitric oxide donating polymer of claim 1 wherein the glass transition temperature is between −30 and 150 C.

8. A medical device having a coating comprised of said nitric oxide donating polymer of claim 1.

9. The nitric oxide donating polymer of claim 8 wherein said implantable medical device is selected from the group consisting of vascular stents, shunts, vascular grafts, stent grafts, heart valves, catheters, pacemakers, pacemaker leads, bile duct stents and defibrillators.

10. The nitric oxide donating polymer according to claim 8 wherein said polymer can release at least one drug in addition to nitric oxide.

11. The nitric oxide donating polymer according to claim 10 wherein said at least one drug is selected from the group consisting of anti-proliferatives, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids.

12. The nitric oxide donating polymer according to claim 10 wherein said drug comprises at least one compound selected from the group consisting of sirolimus (rapamycin), tacrolimus (FK506), everolimus (certican), temsirolimus (CCI-779) and zotarolimus (ABT-578).

13. A medical device having a structure wherein said structure comprising said nitric oxide donating polymer of claim 1.

14. The nitric oxide donating polymer of claim 13 wherein said implantable medical device is selected from the group consisting of vascular stents, shunts, vascular grafts, stent grafts, heart valves, catheters, pacemakers, pacemaker leads, bile duct stents and defibrillators.

15. The nitric oxide donating polymer according to claim 13 wherein said polymer can release at least one drug in addition to nitric oxide.

16. The nitric oxide donating polymer according to claim 15 wherein said at least one drug is selected from the group consisting of anti-proliferatives, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids.

17. The nitric oxide donating polymer according to claim 15 wherein said drug comprises at least one compound selected from the group consisting of sirolimus (rapamycin), tacrolimus (FK506), everolimus (certican), temsirolimus (CCI-779) and zotarolimus (ABT-578).
